(19) United States
(12) Patent Application Publication
(10) Pub. No.: US 2021/0353951 A1
(43) Pub. Date: Nov. 18, 2021

(54) BRAIN STIMULATION DEVICE

(71) Applicant: BBB INC., Seongnam-Si Gyeonggi-Do (KR)

(72) Inventors: Jae Kyu CHOI, Seoul (KR); Eun Jong CHOI, Daejeon (KR); Kyongsik YUN, Seoul (KR); Hyun Doo HWANG, Seongnam-Si (KR)

(73) Assignee: BBB INC., Seongnam-Si Gyeonggi-Do (KR)

(21) Appl. No.: 17/384,541

(22) Filed: Jul. 23, 2021

Related U.S. Application Data
(63) Continuation of application No. PCT/KR2019/001704, filed on Feb. 12, 2019.

(30) Foreign Application Priority Data
Jan. 24, 2019 (KR) 10-2019-0009225

(51) Int. Cl. A61N 2/00; A61N 2/02

(52) U.S. Cl. CPC A61N 2/006; A61N 2/02

(57) ABSTRACT

The present invention relates to a brain stimulation device, including: an electrode unit constituted by one or more electrodes and configured to move back and forth to a head of a user to fix the electrodes onto the head of the user; an auxiliary pressing unit configured to move back and forth to the head of the user to fix the electrodes onto the head of the user; and a donut type support unit having a donut type inner side on which the electrode unit and the auxiliary pressing unit are disposed and which surrounds the head, therefore a user can conveniently wear the brain stimulation device without a complicated manipulation for closely attaching the electrodes to his/her head.

### CROSS-REFERENCE TO RELATED APPLICATION

The present application is a continuation of International Patent Application No. PCT/KR2019/001704, filed Feb. 12, 2019, which is based upon and claims the benefit of priority to Korean Patent Application No. 10-2019-0009225, filed on Jan. 24, 2019. The disclosures of the above-listed applications are hereby incorporated by reference herein in their entirety.

### TECHNICAL FIELD

The present invention relates to a brain stimulation device, and more particularly, to a brain stimulation device which a user can conveniently wear without a complicated manipulation for closely attaching an electrode to his/her head.

### BACKGROUND ART

A brain as an internal organ of a human head is a highest central organ of a nervous system, and is divided into cerebrums, cerebellum, midbrain, pons, and medulla. Further, the brain generates a brainwave which is a signal indicating the sum of neural activity levels measured in the epidermis of the brain.

Examples of a method for measuring a state of the brain include an electroencephalogram inspection (EEG) that inspects the state of the brain by measuring the brainwave received from an electrode by mounting a pad having the electrode on the epidermis, a CT inspection that inspects the state of the brain by performing tomography of the brain at various angles by using radiation or ultrasonic waves, an MRI inspection that photographs the brain by magnetic resonance, and the like.

FIG. 1 illustrates an example of a conventional EEG inspection.

In the EEG inspection, a swimming cap type elastic measuring device is worn and the electrodes are attached one by one, which requires a long measurement preparation time.

Meanwhile, various concepts are known in the field of neural stimulation of brain structures, and brain stimulation which stimulates the brain to achieve a predetermined object is largely divided into invasive brain stimulation and non-invasive brain stimulation.

The invasive brain stimulation is a method that penetrates an electrode into the brain through surgery and applies an electrical signal thereto, and the non-invasive brain stimulation is a method that achieves a predetermined effect by stimulating the brain without invading an electrode into cranium.

Examples of specific brain stimulation include deep electrical stimulation, transcranial magnetic stimulation (TMS), and transcranial electrical stimulation (TES), and examples of the transcranial electrical stimulation (TES) include transcranial direct current stimulation (tDCS), transcranial alternating current stimulation (tACS), and transcranial random noise stimulation (tRNS).

FIG. 2 illustrates an example of conventional transcranial direct current stimulation (tDCS).

In the case of the transcranial direct current stimulation (tDCS), a weak continuous direct current is applied through two large-sized electrodes provided on the epidermis. This causes a small change in membrane potentials of nerve cells of a cortex and a change in firing rate, and as a result, an excitement level of the nerve cell is influenced.

Referring to FIG. 2, an electrode is fixed by using a headband. Since people have different head shapes and sizes, a band which is adjustable in length or stretchable should be used.

However, such a swimming cap or headband type brain stimulation device is vulnerable to contamination and weak in durability, and requires a long measurement preparation time.

### Technical Problem

Therefore, an object to be achieved by the present invention is to provide a brain stimulation device which a user can conveniently wear without a complicated manipulation for closely attaching an electrode to his/her head.

### Technical Solution

In order to achieve the object, the present invention provides a brain stimulation device including: an electrode unit constituted by one or more electrodes and configured to move back and forth to a head of a user to fix the electrodes onto the head of the user; an auxiliary pressing unit configured to move back and forth to the head of the user to fix the electrode unit onto the head of the user; and a donut type support unit having a donut type inner side on which the electrode unit and the auxiliary pressing unit are disposed and which surrounds the head of the user.

According to an embodiment of the present invention, the electrodes may be constituted by two electromagnets, and each electromagnet may be changed to the N pole and the S pole over time to apply a stimulus to the head of the user.

Meanwhile, when the two electromagnets constituting the electrodes are the same stimulus, the electromagnets may stimulate a deep place of the head of the user, and when the two electromagnets constituting the electrodes are different stimuli, the electromagnets may stimulate a shallow place of the head of the user.

According to another embodiment of the present invention, each of the electrodes of the electrode unit may be located in a cap expanded by air pressure, and the cap may be expanded to closely attach the electrode unit to the head.

Further, a pressure sensor may be provided on one end of the electrode unit contacting the head, and the electrode unit may move to the head until a pressure of the pressure sensor becomes a predetermined pressure or higher.

According to still another embodiment of the present invention, the brain stimulation device may further include an electrode connected to the donut type support unit by a band and disposed inside the donut type support unit, in which when the donut type support unit surrounds the head, the electrodes may be closely attached onto the head.

### Advantageous Effects

According to the present invention, a user can conveniently wear a brain stimulation device a complicated manipulation for closely attaching an electrode to his/her head.

Further, according to the present invention, the electrode is closely attached to the head, and then pressed through pneumatic expansion, such that the brain stimulation device can be worn regardless of the size of a user's head.

### BRIEF DESCRIPTION OF DRAWINGS

FIG. 1 illustrates an example of a conventional EEG inspection.

FIG. 2 illustrates an example of conventional transcranial direct current stimulation (tDCS).

FIG. 3 is a configuration diagram of a brain stimulation device according to an embodiment of the present invention.

FIG. 4 illustrates a polarity change pattern when polarities of electrodes constituting an electrode unit 310 are constituted by two polarities.

FIGS. 5A-5D illustrate directions of magnetic fluxs of the electrode unit 310 for each mode.

FIG. 6 is a configuration diagram of a brain stimulation device according to another embodiment of the present invention.

FIG. 7 is a configuration diagram of a brain stimulation device according to still another embodiment of the present invention.

FIG. 8 illustrates a state in which a brain stimulation device according to an embodiment of the present invention is worn on a head of a user.

### BEST MODE

In order to achieve the object, the present invention provides a brain stimulation device including: an electrode unit constituted by one or more electrodes and configured to move back and forth to a head of a user to fix the electrodes onto the head of the user; an auxiliary pressing unit configured to move back and forth to the head of the user to fix the electrode unit onto the head of the user; and a donut type support unit having a donut type inner side on which the electrode unit and the auxiliary pressing unit are disposed and which surrounds the head of the user.

### DETAILED DESCRIPTIONS

Hereinafter, embodiments of the present invention will be described in detail with reference to the accompanying drawings so that those skilled in the art can easily carry out the present invention. However, the embodiments are provided for more specifically describing the present invention, and it will be obvious to those skilled in the art that the scope of the present invention is not limited thereto.

The configuration of the present invention for clarifying a solution to the problem to be solved by the present invention will be described in detail with reference to the accompanying drawings based on embodiments of the present invention. It is to be noted that in assigning reference numerals to the components of the drawings, the same reference numerals are assigned to the same components even though the same components are illustrated on different drawings, and components of other drawings may be cited when a certain drawing needs to be explained. In addition, when it is determined that detailed descriptions of publicly-known functions or configurations related to the present invention and the other general matters may unnecessarily obscure the subject matter of the present invention in describing the operating principle for the embodiment of the present invention in detail, the detailed descriptions will be omitted. Further, throughout the specification, when it is described that a part is “connected” with another part, it not only means that the parts are “directly connected” to each other, and but also means that the parts are “indirectly connected” to each other with another element interposed therebetween. In this specification, the singular form also includes the plural form, unless the context indicates otherwise. The term “comprise” or “comprising” used in the specification indicates the inclusion of a stated component, step, operation or element, but does not exclude the presence or addition of one or more other components, steps, operations, or elements.

FIG. 3 is a configuration diagram of a brain stimulation device according to an embodiment of the present invention.

Referring to FIG. 3, the brain stimulation device according to the present embodiment is constituted by an electrode unit 310 and a donut type support unit 320.

The electrode unit 310 is constituted by four or more electrodes, and in order to closely attach the electrodes to a head of a user, the electrodes move back and forth from an inner side of the donut type support unit 320 to the head.

Referring to FIG. 3(a), four electrodes are disposed on the inner side of the donut type support unit 320 and while moving back and forth, the electrodes are closely attached to the head.

FIG. 3(b) illustrates a state in which fourth electrodes are inserted into the inside of the donut type support unit 320.

The respective electrodes of the electrode unit 310 are located in a cap expanded by air pressure, and the electrode unit 310 may be closely attached to the head by moving the electrodes back and forth and expanding the cap.

The donut type support unit 320 has a donut type inner side on which the electrode unit 310 and an auxiliary pressing unit 400 are disposed and which surrounds the head.

Further, as another embodiment, a pressure sensor may be provided on one end of the electrode unit 310 contacting the head, and the electrode unit 310 may move to the head until a pressure of the pressure sensor becomes a predetermined pressure or higher.

The respective electrodes constituting the electrode unit 310 may be closely attached to the head to apply the pressure to the head, by moving the electrodes and closely attaching the electrodes to the head until a pressure measured by the pressure sensor provided in each of the electrodes becomes a predetermined threshold or higher, or by increasing the air pressure in the cap including the electrodes.

Air may be supplied to or discharged from the electrode unit 310 by using an air pump, such that back and forth movement of the electrode unit 310 or expansion or contraction of the cap is made. In order to closely attach the electrode unit 310 to the head, the air may be supplied to the electrode unit 310 through the air pump, and simultaneously, an air valve may be closed, and an operation of the air pump may be stopped and the air valve may be opened in order to return the electrode unit 310 to its original position. The cap including the electrodes may also be expanded and contracted by using the air pump and the air valve.

As another embodiment, a user may manually determine the back and forth movement of the electrodes of the electrode unit 310 and the increase or decrease of the air pressure of the electrode unit 310.

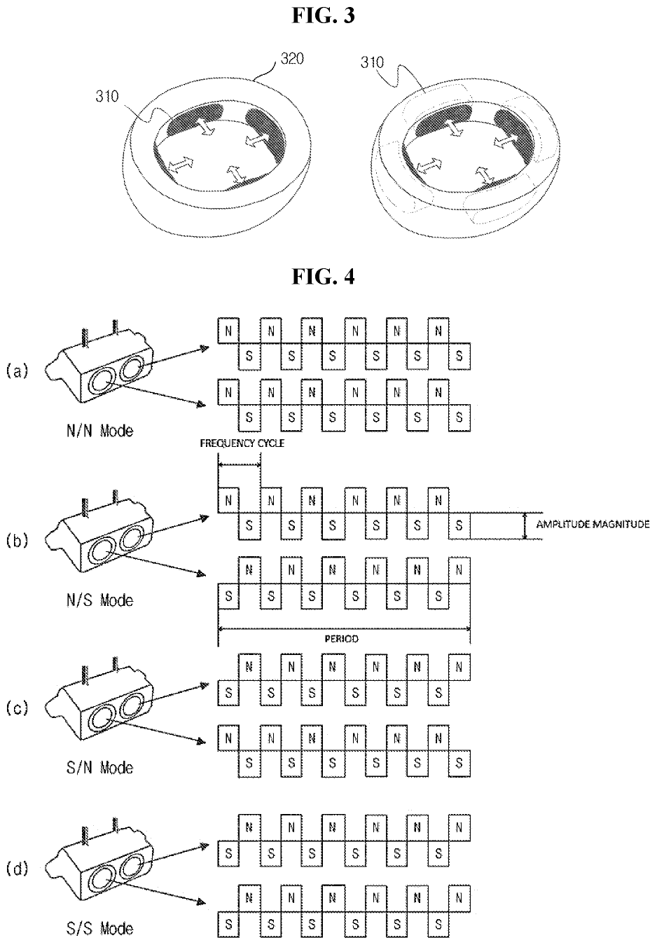

FIG. 4 illustrates a polarity change pattern when polarities of electrodes constituting the electrode unit 310 are constituted by two polarities.

The electrodes constituting the electrode unit 310 may be configured by electromagnets having magnetic properties of the N pole and the S pole by a flow of current. The electrode unit 310 is constituted by one or more electromagnets, but the embodiment of the present invention illustrates a case where two electromagnets are provided.

The electrodes illustrated in FIG. 4 are constituted by two electromagnets, and each electromagnet is changed to the N pole and the S pole over time, thereby applying a stimulus to the head of the user.

When two electromagnets constitute the electrode unit 310, the magnetic property of each electromagnet may be changed to an N/N mode, an N/S mode, an S/N mode, and an S/S mode.

(a) FIG. 4 illustrates the N/N mode, (b) of FIG. 4 illustrates the N/S mode, (c) of FIG. 4 illustrates the S/N mode, and (d) of FIG. 4 illustrates the S/S mode.

Various modified embodiments of each mode are available according to an amplitude magnitude, a stimulation period, and a frequency cycle. Table 1 shows the stimulation period, a frequency, an amplification magnitude, and an available mode in each electrode.

TABLE 1

| | Front | Rear | Left | Right |
|---|---|---|---|---|
| | electrode | electrode | electrode | electrode |
| Stimulation | 1.0 to 120 min | | | |
| period | | | | |
| Frequency | 0.1 to 3,000 Hz | | | |
| Amplification | 0.1 to | 0.1 to | 0.1 to | 0.1 to |
| magnitude | 100 mT | 100 mT | 100 mT | 100 mT |
| Mode | N/N, N/S, | N/N, N/S, | N/N, N/S, | N/N, N/S, |
| | S/N, S/S | S/N, S/S | S/N, S/S | S/N, S/S |

In the case of the N/N mode or the S/S mode, the magnitude of the magnetic flux output from the electrode unit 310 may be enhanced, such that the magnetic flux may stimulate a deep place of the head, and in the case of the N/S mode or the S/N mode, the magnetic flux output from the N pole of the electrode unit 310 may be immediately input into the S pole located next to the N pole, such that the magnetic flux may stimulate a shallow place of the head. That is, when two electromagnets constituting the electrodes have the same polarity, the electromagnets may stimulate the deep place of the head of the user, and when two electromagnets constituting the electrodes have a different polarity, the electromagnets may stimulate the shallow place of the head of the user.

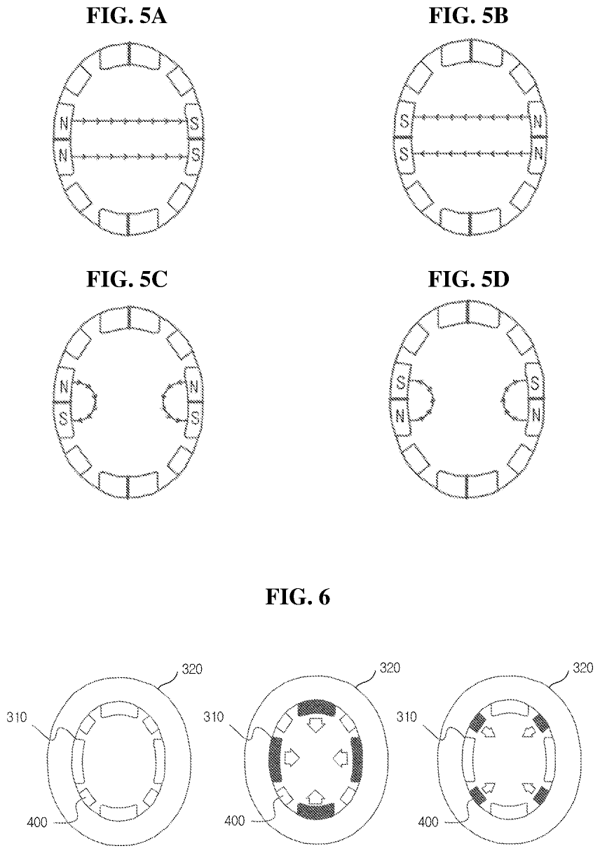

FIGS. 5A-5D illustrate directions of magnetic fluxs of the electrode unit 310 for each mode.

FIGS. 5A and 5B illustrate a magnetic flux connected from the electrodes in the N/N mode to the electrodes in the S/S mode.

FIGS. 5C and 5D illustrate that a magnetic flux output from the N pole is connected to the S pole located next to the N pole in the N/S mode or the S/N mode.

Referring to FIGS. 5A-5D, it can be seen that in the N/N mode or the S/S mode, the magnetic flux reaches the deep place of the head to stimulate the deep place of the head, and it can be seen that in the N/S mode or the S/N mode, a sufficient magnetic flux may not reach the deep place of the head, and the magnetic flux is formed in the shallow place of the head.

FIG. 6 is a configuration diagram of a brain stimulation device according to another embodiment of the present invention.

Referring to FIG. 6, the brain stimulation device according to the present embodiment is constituted by the electrode unit 310, the donut type support unit 320, and the auxiliary pressing unit 400.

The descriptions of the electrode unit 310 and the donut type support unit 320 are duplicated with those in FIG. 3 and thus will be omitted.

The auxiliary pressing unit 400 is located between the electrodes of the electrode unit 310, and moves back and forth to the head from the inner side of the donut type support unit 320 in order to closely attach the electrode unit 310 to the head of the user.

Respective individual pressing units of the auxiliary pressing unit 400 are located in the cap expanded by the air pressure, and the auxiliary pressing unit 400 may be more closely attached to the head by moving the individual pressing units back and forth, and expanding the cap, as compared to when only the electrode unit 310 is used. The brain stimulation device according to the present invention may be more stably fixed to the head through the auxiliary pressing unit 400.

Further, as another embodiment, a pressure sensor may be provided on one end of the auxiliary pressing unit 400 contacting the head, and the auxiliary pressing unit 400 may move to the head until a pressure of the pressure sensor becomes a predetermined pressure or higher.

Until the pressure measured by the pressure sensor provided in each of the individual pressing units becomes a predetermined threshold or more, the respective individual pressing units constituting the auxiliary pressing unit 400 may be closely attached to the head to apply the pressure to the head. In this case, it is possible to further closely attach the individual pressing units or increase the air pressure in order to increase the pressure.

Meanwhile, the user may manually determine the back and forth movement of the individual pressing units of the auxiliary pressing unit 400, and the increase of the air pressure.

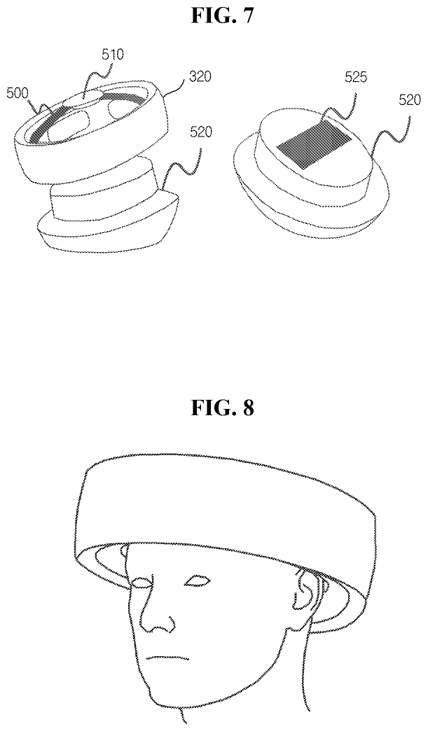

FIG. 7 is a configuration diagram of a brain stimulation device according to still another embodiment of the present invention.

The brain stimulation device illustrated in FIG. 7 is configured to further include a connection unit 500, an upper electrode 510, and a cradle 520.

The connection unit 500 connects the upper electrode 510 and the donut type support unit 320. The connection unit 500 is formed by a stretchable band, and when the head of the user and the upper electrode 510 are in contact with each other, the connection unit 500 changes a position of the upper electrode 510 in a predetermined range and enables the brain stimulation device to be stably worn on the head.

When the user wears the brain stimulation device, the upper electrode 510 is an electrode that applies a stimulus to an upper portion of the head. The upper electrode 510 is connected to the donut type support unit 320 by the connection unit 500.

The cradle 520 is a device that keeps the brain stimulation device when the user does not use the brain stimulation device. Since the donut type support unit 320 is a donut type, a protrusion 522 is formed in the cradle 520 so that the brain stimulation device is suspended through an empty space at the center of the donut type support unit 320.

The cradle 520 includes a display unit 525, and the display unit 525 displays back and forth movement distances of the electrode unit 310 and the auxiliary pressing unit 400, the air pressure, etc. Further, the display unit 525 is configured by a touch screen, and the brain stimulation device may be controlled by touching the display unit 525.

FIG. 8 illustrates a state in which a brain stimulation device according to an embodiment of the present invention is worn on a head.

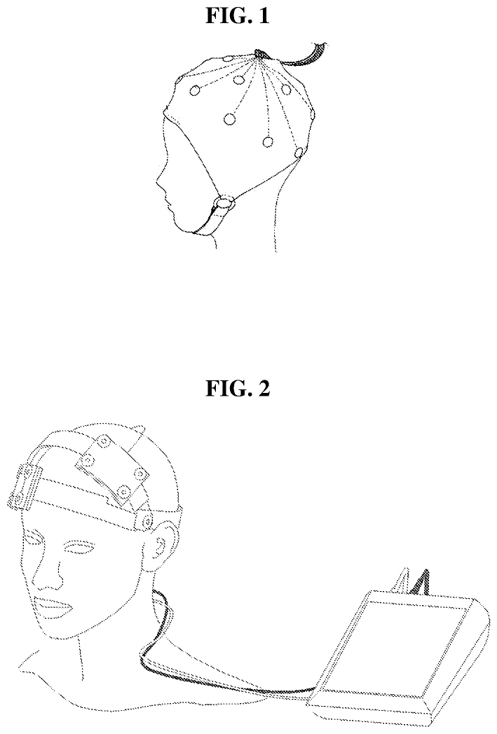

Referring to FIG. 8, it can be seen that the brain stimulation device can be conveniently worn like the swimming cap type illustrated in FIG. 1, and can closely attach the electrodes to the head unlike the headband type illustrated in FIG. 2.

By using the brain stimulation device according to the present invention, transcranial magnetic stimulation (TMS) and repetitive transcranial magnetic stimulation (rTMS) may be performed.

When the brain is stimulated, the brain exhibits an excitability or inhibiting effect through a series of complicated processes, and a technical principle of the transcranial magnetic stimulation (TMS) is to cause a magnetic field which is abruptly changed by making a short and strong current flow on a coil, and such a magnetic field causes an electric field again to excite nerve cells of the cerebrum when stimulating an upper portion of the epidermis of a person.

The repetitive transcranial magnetic stimulation (rTMS) is a method that causes an evoked potential by continuous magnetic stimuli to activate the nerve cells of a cerebral cortex. When the continuous stimuli are applied with various frequencies and strengths, the continuous stimuli may cause the excitability or inhibiting effect for the cerebral cortex, and change a cerebral cortex activity in a short or long term. A low-frequency stimulus of one time per second, i.e., 1 Hz stimulus inhibits the activity of a motor cortex and a high-frequency stimulus of 10 times or 20 times per second, i.e., 10 Hz or 20 Hz stimulus increases the cerebral cortex activity in a short term, which makes it possible to provide various effects. By using such effects, the brain stimulation device may be attempted to a variety of clinical diseases including stroke, depression, Parkinson's disease, epilepsy, pain, and the like.

As described above, the present invention has been described with reference to specified matters such as detailed components, and limited embodiments and drawings, but the embodiments are just provided to assist more overall understanding of the present invention, the present invention is not limited to the embodiments, and various modifications and changes can be made by those skilled in the art from such descriptions.

Accordingly, the spirit of the present invention should not be defined as being limited to the described embodiments, and it should be appreciated that claims to be described below and all which are equivalent or equivalently modified to the claims are included in the scope of the present invention.

1. A brain stimulation device comprising:
an electrode unit constituted by one or more electrodes and configured to move back and forth to a head of a user to fix the electrodes onto the head of the user;
an auxiliary pressing unit configured to move back and forth to the head of the user to fix the electrode unit onto the head of the user; and
a donut type support unit having a donut type inner side on which the electrode unit and the auxiliary pressing unit are disposed and which surrounds the head of the user.

2. The brain stimulation device of claim 1, wherein the electrodes are constituted by two electromagnets, and each electromagnet is changed to the N pole and the S pole over time to apply a stimulus or multiple stimuli to the head of the user.

3. The brain stimulation device of claim 1, wherein each of the electrodes of the electrode unit is located in a cap expanded by air pressure, and
the electrode unit is closely attached to the head by expanding the cap.

4. The brain stimulation device of claim 1, wherein a pressure sensor is provided on one end of the electrode unit contacting the head, and
the electrode unit moves to the head until a pressure of the pressure sensor becomes a predetermined pressure or higher.

5. The brain stimulation device of claim 1, further comprising:
one or more electrodes connected to the donut type support unit by a band and disposed inside the donut type support unit,
wherein when the donut type support unit surrounds the head, the electrodes are closely attached onto the head.

6. The brain stimulation device of claim 2, wherein when the two electromagnets constituting the electrodes have the same polarity, the electromagnets stimulate a deep place of the head of the user, and when the two electromagnets constituting the electrodes have a different polarity, the electromagnets stimulate a shallow place of the head of the user.
